(19) United States
(12) Patent Application Publication
(10) Pub. No.: US 2024/0391888 A1
(43) Pub. Date: Nov. 28, 2024

(54) METHODS OF CARBONYLATING 2,2,3-TRIALKYLOXIRANES AND PRODUCTS THEREOF

(71) Applicant: CORNELL UNIVERSITY, Ithaca, NY (US)

(72) Inventors: Geoffrey W. Coates, Lansing, NY (US); Aran Hubbell, Ithaca, NY (US)

(21) Appl. No.: 18/669,435

(22) Filed: May 20, 2024

Related U.S. Application Data
(60) Provisional application No. 63/467,392, filed on May 18, 2023.

(51) Int. Cl. C07D 305/12; B01J 31/14; B01J 31/20; C08G 63/08

(52) U.S. Cl. CPC C07D 305/12; B01J 31/143; B01J 31/20; C08G 63/08

(57) ABSTRACT

Methods of producing one or more trisubstituted lactone(s) comprising contacting a reaction mixture comprising one or more 2,2,3-trisubstituted epoxide(s) and one or more catalyst(s) with carbon monoxide. A catalyst may comprise a cationic Lewis acid and an anionic metal carbonyl. Methods may be regioselective and/or stereoselective. Trisubstituted lactones, which may be 3,3,4-trisubstituted β-lactones. Compositions comprising trisubstituted lactone(s). Polymers formed from trisubstituted lactone(s).

### CROSS REFERENCE TO RELATED APPLICATIONS

This application claims priority to U.S. Provisional Application No. 63/467,392, filed on May 18, 2023, the entire disclosure of which is hereby incorporated by reference.

### STATEMENT REGARDING FEDERALLY SPONSORED RESEARCH

This invention was made with government support under grant no. DE-FOA-0002414 awarded by the Department of Energy. The government has certain rights in the invention.

### BACKGROUND OF THE DISCLOSURE

Poly(pivalolactone), an aliphatic polyester with the structure [—OCH₂C(Me)₂C(═O)—]ₙ, is a semicrystalline thermoplastic that has excellent mechanical properties as well as good thermal stability. A related polymer with the structure [—OCHMeC(Me)₂C(═O)—]ₙ, even as an atactic material, has been shown to be semi-crystalline with a melting temperature of 139° C., along with excellent thermal stability, up to ca. 270° C. Unfortunately, the polymer is synthesized from the ring-opening of 3,3,4-trimethyl-2-oxetanone, a beta-lactone synthesized in modest purity (85.1-98.4) via the cycloaddition of acetaldehyde and dimethylketene, the latter being a relatively expensive and unstable feedstock.

### SUMMARY OF THE DISCLOSURE

The present disclosure provides, inter alia, lactones and methods for producing lactones.

In an aspect, the present disclosure provides methods for producing trisubstituted lactones. The method may comprise contacting a reaction mixture comprising one or more 2,2,3-trisubstituted epoxide(s) and one or more catalyst(s) with carbon monoxide.

In an aspect, the present disclosure provides trisubstituted lactones. The trisubstituted lactones may comprise 3,3,4-trisubstituted β-lactones, such as, for example, 3,3,4-trimethyl-2-oxetanone and/or 4-ethyl-3,3-dimethyloxetan-2-one.

In an aspect, the present disclosure provides compositions comprising trisubstituted lactones. The trisubstituted lactones may be produced by a method of the present disclosure.

In an aspect, the present disclosure provides polymers. The polymers may be formed by the trisubstituted lactones of the present disclosure.

The present disclosure satisfies, inter alia, the need for improved methods of making trisubstituted lactones. In particular, the present disclosure provides trisubstituted lactones that are useful, inter alia, to make polymers.

### BRIEF DESCRIPTION OF THE FIGURES

For a fuller understanding of the nature and objects of the disclosure, reference should be made to the following detailed description taken in conjunction with the accompanying figures.

FIG. 1 shows examples of carbonylation catalysts.

FIG. 2 shows examples of methods and resulting lactone products.

FIG. 3 shows an example of a method, the resulting lactone product, and the polymerized form of the lactone product.

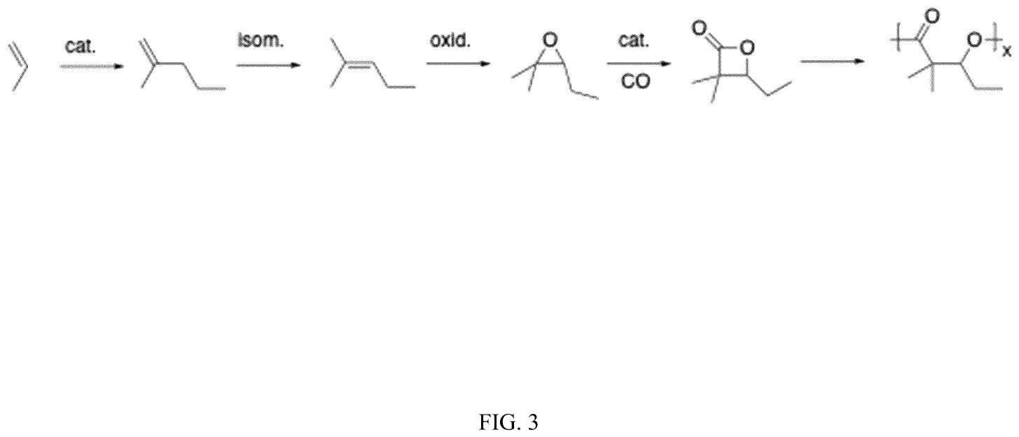

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise stated, “about,” “approximately,” “substantially,” or the like, when used in connection with a measurable variable such as, for example, a parameter, an amount, a temporal duration, or the like, are meant to encompass variations of, for example, a specified value including, for example, those within experimental error (which can be determined by for example, a given data set, an art accepted standard, and/or with a given confidence interval (e.g. 90%, 95%, or more confidence interval from the mean), such as, for example, variations of +/−10% or less, +/−5% or less, +/−1% or less, and +/−0.1% or less of and from the specified value) or to encompass alternatives to the members of the list that would be recognized by one of ordinary skill in the art as alternatives, where the members and the alternatives may define a genus or sub-genus, insofar as such variations are appropriate to perform in the context of the disclosure. As used herein, unless otherwise stated, the terms “about,” “approximate,” “at or about,” “substantially,” and “˜” can mean that the amount or value in question can be the exact value or a value that provides equivalent results or effects as recited in the sample claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error, and the like, and other factors known to those of skill in the art such that, for example, equivalent results, effects, or the like are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” “at or about,” or “˜” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also, unless otherwise stated, include individual values (e.g., about 1%, about 2%, about 3%, and about 4%) and the sub-ranges (e.g., about 0.5% to about 1.1%; about 0.5% to about 2.4%; about 0.5% to about 3.2%, and about 0.5% to about 4.4%, and other possible sub-ranges) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further disclosure. For example, if the value “about 10” is disclosed, then “10” is also disclosed.

As used herein, unless otherwise stated, the term “group” or “moiety” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups or moieties include:

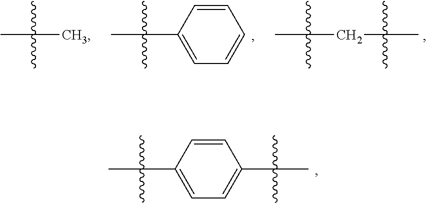

and the like.

As used herein, unless otherwise indicated, the term “alkyl group” refers to branched or unbranched, linear saturated hydrocarbon groups. In various examples, an alkyl group is a cyclic alkyl group (e.g., a carbocycle or the like). Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. In various examples, an alkyl group is a C₁ to C₃₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). Alkyl groups may be unsubstituted or substituted with one or more substituent(s). Examples of substituents include, but are not limited to, halide groups (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxy groups, alkoxide groups, amine groups, nitro groups, carboxylate groups, carboxylic acids, ether groups, alcohol groups, alkyne groups (e.g., acetylenyl groups and the like), structural analogs thereof, and the like, and any combination thereof.

As used herein, unless otherwise indicated, the term “aryl group” refers to aromatic groups and partially aromatic carbocyclic groups. In various examples, an aryl group refers to C₅ to C₃₀ aromatic groups or partially aromatic carbocyclic groups, including all integer numbers of carbons and ranges of numbers of carbons therebetween, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). In various examples, an aryl group is an aromatic group. In various examples, an aryl group is a polyaryl group, such as, for example, a fused ring, a biaryl group, or any combination thereof. Aryl groups may be unsubstituted or substituted with one or more substituent(s). Examples of substituents include, but are not limited to, halides (—F, —Cl, —Br, and —I), aliphatic groups (e.g., alkyl groups, alkenyl groups, alkynyl groups, and the like), aryl groups, alkoxides, carboxylates, carboxylic acids, ether groups, structural analogs thereof, and the like, and any combination thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, furanyl groups, benzofuranyl groups, indolyl groups, imidazolyl groups, benzimidazolyl groups, pyridinyl groups, structural analogs thereof, and the like.

As used herein, unless otherwise indicated, the term “structural analog” refers to any compound (e.g., lactone, epoxide, catalyst, catalyst precursor, solvent, any portion thereof (e.g., ligand, functional group, or the like), or the like, or any combination thereof), that can be envisioned to arise from an original compound) if one atom or group of atoms, functional groups, or substructures is replaced with another atom or group of atoms, functional groups, substructures, or the like. In various examples, the term “structural analog” refers to any group that is derived from an original compound by a chemical reaction, where the compound is modified or partially substituted such that at least one structural feature of the compound or group is retained.

The present disclosure discloses, inter alia, methods of producing carbonyl compounds (e.g., carbonyl containing compounds). The present disclosure also provides carbonyl compounds (e.g., carbonyl containing compounds). In various examples, a carbonyl compound (e.g., carbonyl containing compound) comprises or is a lactone (e.g., a trisubstituted lactone, such as, for example, a trisubstituted β-lactone).

In an aspect, the present disclosure provides methods of producing lactone compounds. A method may produce lactone compounds, such as, for example 3,3,4-trisubstituted β-lactone(s) (such as, for example, 3,3,4-trialkyl-2-oxetanone(s) (e.g., 3,3,4-trimethyl-2-oxetanone, 4-ethyl-3,3-dimethyloxetan-2-one, and the like) and the like and any combination thereof) and the like. In various examples, a method is based on carbonylation at the more substituted carbon (carbon position) of a cyclic ether. In various examples, a method comprises catalytic, regioselective carbonylation of, for example, 2,2,3-trimethyloxirane to produce a desired 3,3,4-trimethyl-2-oxetanone in desirable yield and/or purity. In various examples, a method comprises regioselective carbonylation of, for example, 3-ethyl-2,2-dimethyloxirane to produce a desired 4-ethyl-3,3-dimethyloxetan-2-one in desirable yield and/or purity. It is expected the instant carbonylation methods can also be applied to other 2,2,3-trialkyloxiranes. Non-limiting examples of methods of producing trisubstituted lactone compounds are provided herein.

It was unexpectedly observed that the carbonylation of 2,2,3-trimethyloxirane cleanly yields the desired beta-lactone 3,3,4-trimethyl-2-oxetanone, with little or no appreciable (e.g., observable) amount of the undesired beta-lactone 3,4,4-trimethyl-2-oxetanone. This is in contrast to the related carbonylation of 2,2-dimethyloxirane, which makes mixtures of 3,3-dimethyl-2-oxetanone and 4,4-dimethyl-2-oxetanone, favoring the undesired 4,4-dimethyl-2-oxetanone.

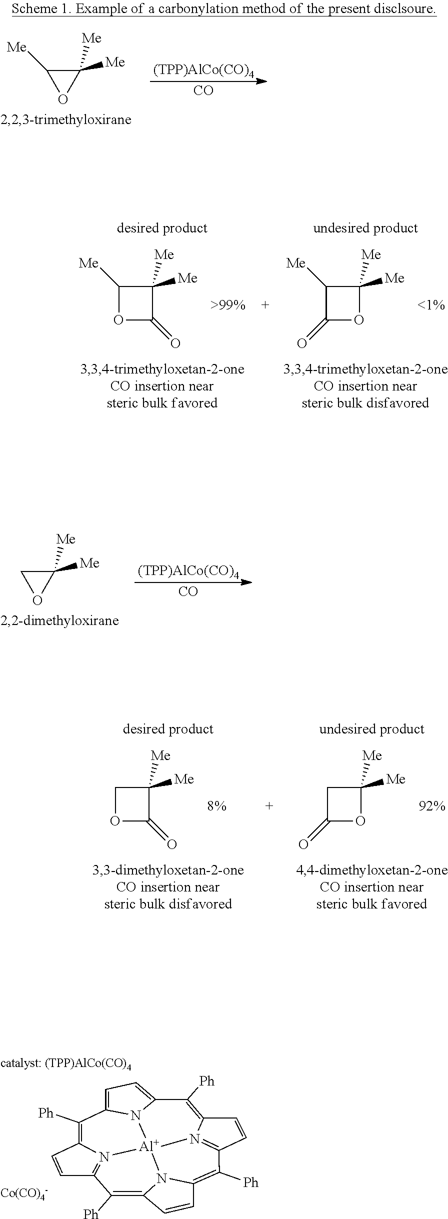

In various examples, a method comprises providing a reaction mixture comprising one or more 2,2,3-trisubstituted epoxide(s) and one or more catalysts(s) (such as, for example, carbonylation catalyst(s) or the like). In various examples, a method further comprises contacting (e.g., pressurizing or the like) the reaction mixture with carbon monoxide (e.g., CO gas), where one or more lactone(s) is/are formed. In various examples, a method comprises contacting a reaction mixture comprising one or more 2,2,3-trisubstituted epoxide(s) and one or more catalyst(s) (such as, for example, carbonylation catalyst(s)) with carbon monoxide (e.g., CO gas), where one or more lactone(s) is/are formed.

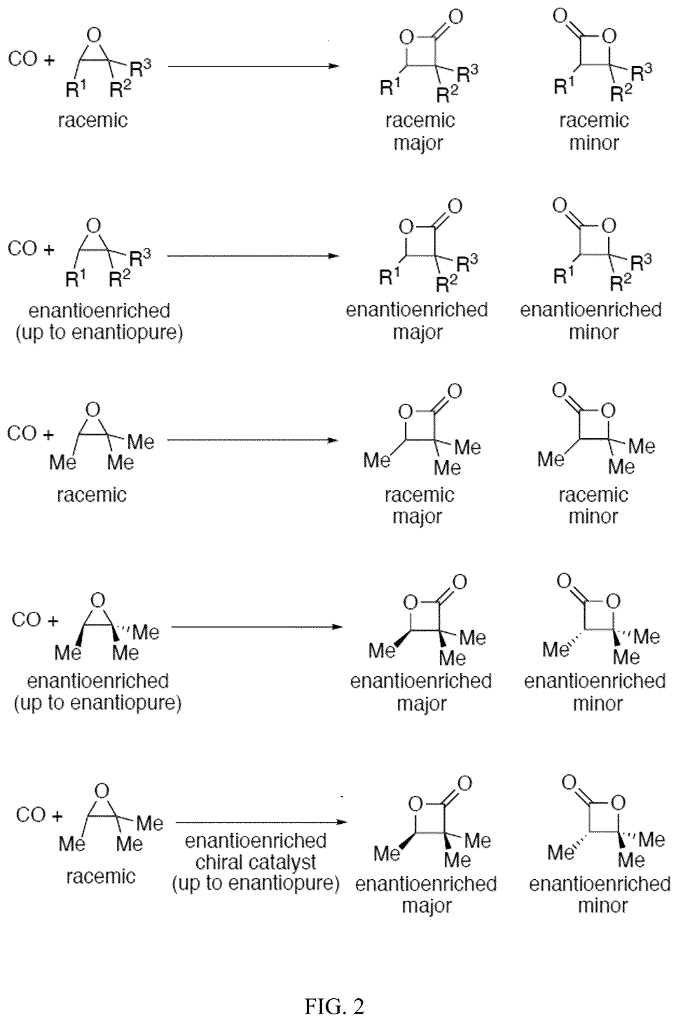

Non-limiting and illustrative examples of methods of the present disclosure and resulting lactone products are shown in FIG. 2. In various examples, the epoxide(s) in example in FIG. 2 is/are replaced with one or more epoxide(s) as described herein and the lactone(s) (e.g., lactone product(s)) are those resulting from carbonylation of epoxide(s) by a method of the present disclosure. In various examples, the epoxide(s) and/or lactone(s) are structural analog(s) of epoxide(s) and/or lactone(s) in FIG. 2. In various examples, a method comprises a catalytic carbonylation and a ring-opening reaction. The catalytic carbonylation reaction occurs first. In this reaction, an epoxide is reacted in the presence of a catalyst to form a lactone (e.g., a trisubstituted lactone).

In various examples, a method provides regioselectivity and/or enantioselectivity (e.g., in trisubstituted lactone(s) produced by the method). In various examples, a regioselective method produces trisubstituted lactone(s) with a ratio of 3,3,4-trisubstituted β-lactone(s) (where the alpha carbon is disubstituted) to 3,4,4-trisubstituted β-lactones (where the beta carbon is disubstituted) of about 50:50 to about >99:<1, including all integer ratio values and ranges therebetween (e.g., about 50:50, greater than about 50:50, about 60:40, about 70:30, about 75:25, about 80:20, about 85:15, about 90:10, about 91:9, about 92:8, about 93:7, about 94:6, about 95:5, about 96:4, about 97:3, about 98:2, about 99:1:>99:1, or about >99:<1). In various examples, an enantioselective method produces lactone(s) with enantioselectivity greater than about 50% to about >99%, including all 0.1% values and ranges therebetween (e.g., about 60% or greater, about 70% or greater, about 80% or greater, about 90% or greater, about 95% or greater, about 98% or greater, about 99% or greater, about 99.5% or greater, or about 99.9% or greater). In various examples, an enantioselective method produces an enantiopure 3,3,4-trisubstituted β-lactone. In various examples, an enantioselective method comprises use of an enantioenriched chiral catalyst or the like.

Various epoxides (e.g., 2,2,3-trisubstituted epoxides) can be used in a method. In various examples, one or more 2,2,3-trisubstituted epoxide(s) comprise(s) one or more stereoisomers, or a mixture (e.g., a racemic mixture) thereof. In various examples, a 2,2,3-trisubstituted epoxide comprises the following structure:

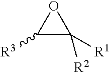

(such as, for example,

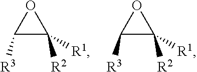

or the like) or the like, or a structural analog thereof, or a stereoisomer thereof, or any combination thereof. In various examples, R¹, R², and R³ are, independently at each occurrence, a substituted or unsubstituted alkyl group. In various examples, R¹, R², and R³ comprise or are the same substituted or unsubstituted alkyl groups (e.g., methyl groups, ethyl groups, or the like). In various examples, any one or more of R¹, R², and R³ comprises or is a different alkyl group from the other R group(s). In various examples, any two of R¹, R², and R³ (e.g., R¹ and R², R¹ and R³, or R² and R³) are bonded (e.g., covalently bonded or the like) such that they form a ring (e.g., a substituted or unsubstituted carbocycle, a substituted or unsubstituted heterocycle, or the like). The substituted or unsubstituted alkyl groups may be linear or branched alkyl groups or cyclic alkyl groups. In various examples, a linear or a branched alkyl group comprises a longest linear chain of 1 to 12 carbons (e.g., 1, 2, 3, 4, 5, 6, 7, 8, 9, 10, 11, or 12 carbons).

In various examples, the 2,2,3-trisubstituted epoxide(s) is/are chosen from:

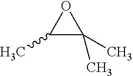

(such as, for example,

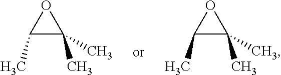

or the like) or the like, or a structural analog thereof, or a stereoisomer thereof, or any combination thereof.

In various examples, the 2,2,3-trisubstituted epoxide(s) is/are chosen from:

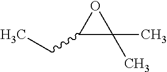

(such as, for example,

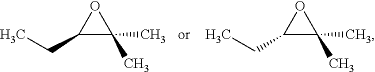

or the like) or the like, or a structural analog thereof, or a stereoisomer thereof, or any combination thereof.

Various catalysts (e.g., carbonylation catalysts or the like) can be used in a method. In various examples, a reaction mixture (e.g., of a method) comprises one or more catalyst(s). In various examples, a reaction mixture comprises a combination of two or more catalysts. In various examples, catalyst(s) are suitable for use in reactions that produce lactones (e.g., trisubstituted lactones or the like) (e.g., carbonylation reactions or the like). In various examples, a catalyst is capable of coordinating to a cyclic substrate, such as, for an example, a cyclic ether (e.g., an epoxide) to form a lactone. In various examples, a catalyst (e.g., carbonylation catalyst or the like) does not produce any or any observable (e.g., by nuclear magnetic resonance spectroscopy or the like) polymerization of the 2,2,3-trisubstituted epoxide(s). Non-limiting examples of catalysts are disclosed herein.

In various examples, one or more or all the catalyst(s) is/are formed in a reaction mixture prior to, concurrently with, or after incorporation (e.g., addition) of the 2,2,3-trisubstituted epoxide(s) to the reaction mixture. In various examples, at least a portion of, substantially all, or all the catalyst(s) are formed in situ (e.g., in situ in a reaction mixture). In various examples, a catalyst is formed from one or more catalytic precursor(s). Non-limiting examples of catalytic precursors include salen compounds, porphyrin compounds, metallocene compounds, structural analogs thereof, and any combination thereof. In various examples, a catalyst is formed from one or more metal source(s). Non-limiting examples of metal sources include cobalt metal sources (e.g., NaCo(CO)₄, Co₂CO₈, Ph₃SiCo(CO)₄, or the like).

In various examples, a catalyst (e.g., carbonylation catalyst or the like) comprises or is a Lewis Acid carbonyl catalyst (e.g., comprising a Lewis Acid cation or the like). Suitable examples of Lewis Acids (e.g., a Lewis Acid cation or the like) are known in the art.

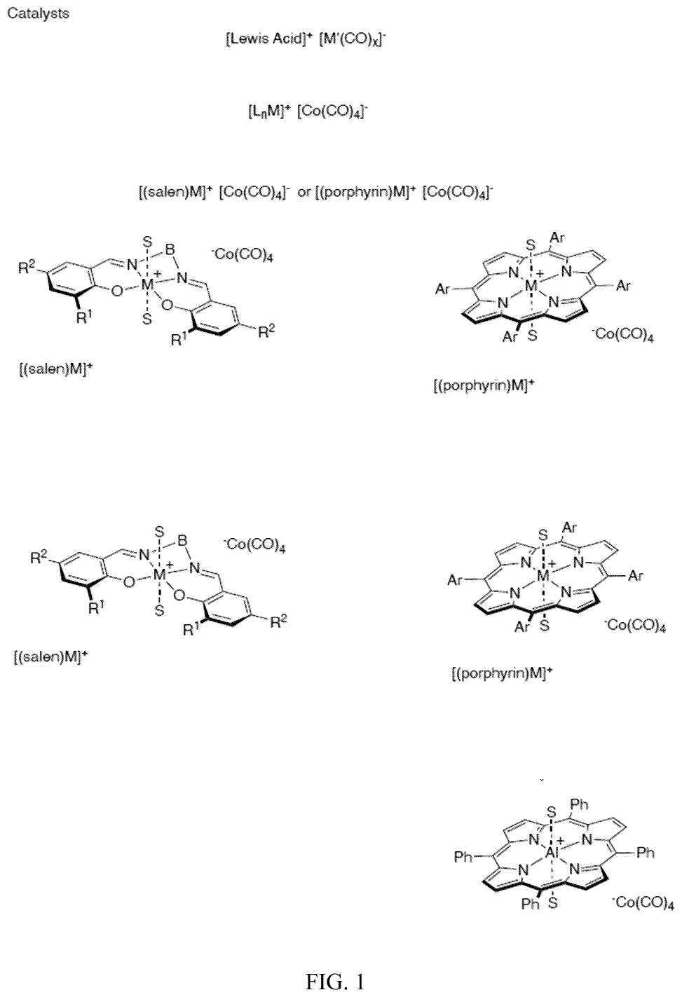

In FIG. 1, M′ of [M′(CO)ₓ]⁻ is chosen from transition metals chosen from transition metals of Groups 4, 5, 6, 7, 8, 9, and 10 of the periodic table of elements (such as, for example, cobalt and the like). In various examples, M′ is a trivalent transition metal of Groups 4, 5, 6, 7, 8, 9, and 10 (such as, for example, cobalt and the like). In various examples, x is an integer that results in formation of a stable anionic transition metal complex. In various examples, x is 4 or the like.

In FIG. 1, M of [LₙM]⁺ is chosen from oxophilic metals and the like. L is a ligand and n is the number of ligands (e.g., 1).

In FIG. 1, M of [(salen)M]⁺ or [(porphyrin)M]⁺ is chosen from oxophilic metals and the like.

In FIG. 1, M of [(salen)M]⁺ is a trivalent metal or the like. S is independently at each occurrence a Lewis basic solvent or the like. B is a carbon-based group (e.g., an alkyl group, an aryl group, or the like). R¹ and R² are substituents (e.g., independently chosen from alkyl groups, alkenyl groups, aryl groups, or the like).

In FIG. 1, M of [(porphyrin)M]⁺ is a trivalent metal or the like. S is i independently at each occurrence a Lewis basic solvent or the like. Ar is an alkyl group or the like).

In FIG. 1, M of [(salen)M]⁺ is Al, Cr, or the like. S is independently at each occurrence an ether (such as, for example, THE or the like). B is 1,2-ethylene group, 1,2-phenylene group, trans-1,2-cyclohexyl group, or the like. R¹ and R² are substituents (such as, for example, independently chosen from halide-substituted, aryl groups, or the like).

In FIG. 1, M of [(porphyrin)M]⁺ is a Al, Cr, or the like. S is independently at each occurrence an ether (such as, for example, THF or the like). Ar is independently at each occurrence a substituted phenyl group (e.g., a 4-chlorophenyl group, a 4-methylphenyl group or the like).

In FIG. 1, S of the specific [(porphyrin)Al]⁺ is THE or the like.

In various examples, a catalyst comprises a Lewis acid (e.g., a chiral Lewis acid or the like) or the like. In various examples, a catalyst comprises a cationic Lewis acid (e.g., a chiral cationic Lewis acid or the like) and an anionic metal carbonyl. In various examples, a catalyst comprises the following formula: [Lewis acid]^{z+} {[QM(CO)ₓ]^{w-}}_{y}, where Q is a ligand and is optional, M is a transition metal chosen from transition metals of Groups 4, 5, 6, 7, 8, 9, and 10 of the periodic table of elements (e.g., cobalt and the like), z is the valence of the Lewis acid and ranges from 1 to 6 (1, 2, 3, 4, 5, or 6), w is the charge of the metal carbonyl and ranges from 1 to 4 (1, 2, 3, or 4), y is a number such that w times y equals z, and x is a number such as to provide a stable anionic metal carbonyl for {[QM(CO)ₓ]^{w-}}_{y} and ranges from 1 to 9 (1, 2, 3, 4, 5, 6, 7, 8, or 9).

In various examples, a catalyst comprises [LA⁺][Co(CO)₄⁻], where [LA⁺] is a Lewis acid capable of coordinating to a cyclic substrate, such as, for an example, a cyclic ether (e.g., an epoxide) to form a lactone. In various examples, catalysts of the present disclosure may be made in situ. In various examples, a metal salt catalyst (e.g., MCI, where M is a metal, such as, for example, aluminum) is added to a reaction mixture with a Co(CO)₄ salt (e.g., NaCo(CO)₄). In various examples, an Al—Cl catalyst is mixed in a solvent with NaCo(CO)₄ and the active complex is formed as depicted in the following schemes:

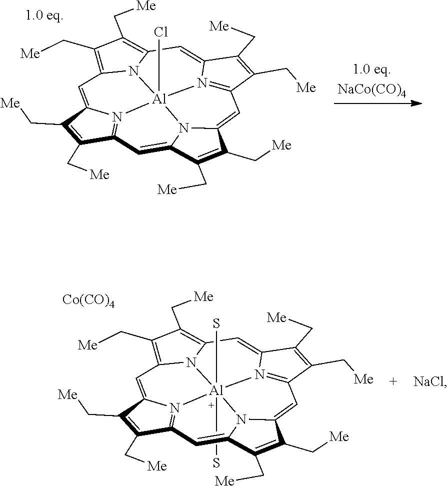

where S is a solvent (e.g., a Lewis-basic solvent), such as, for example, THF.

In various examples, a porphyrin-based Lewis acid catalyst comprises the following structure:

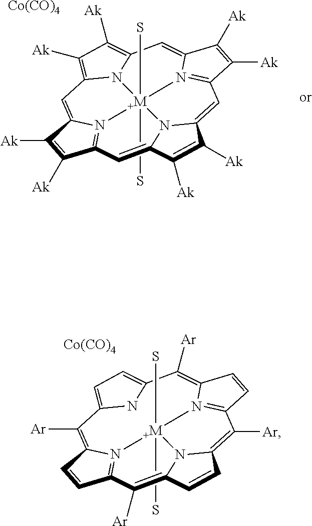

where S is solvent (e.g., THF), M is a metal (e.g., aluminum or chromium), Ak is a substituted linear or branched C₁ to C₁₀ alkyl group or unsubstituted linear or branched C₁ to C₁₀ alkyl group (e.g., methyl, ethyl, propyl, and the like), where each Ak may be the same or different, and Ar is a substituted or unsubstituted aryl group, where each Ar may be the same or different. The aryl groups may be substituted with halogen groups, alkoxy groups (e.g., methoxy groups), or various substituted linear or branched C₁ to C₁₀ alkyl groups or unsubstituted linear or branched C₁ to C₁₀ alkyl groups. In various examples, Ar is chosen from phenyl, p-ClC₆H₄, p-FC₆H₄, p-OMeC₆H₄, and 2,4,6-Me₃C₆H₂. In various examples Ak is ethyl. In various examples, the metal is aluminum.

In various examples, a salph/salen-based Lewis acid catalyst comprises the following structure:

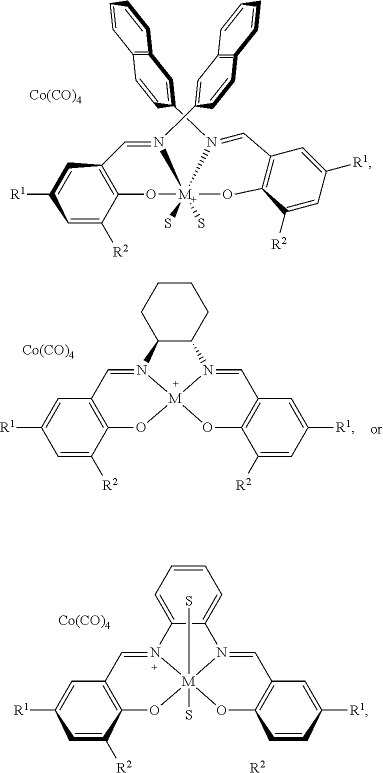

where S is solvent (e.g., THF), M is a metal (e.g., aluminum or chromium), and R¹ and R² are independently chosen from H, alkyl groups, halogen groups, and alkoxide groups. R¹ and R² may be the same or different.

Non-limiting examples of suitable catalysts include N,N′-Bis(3,5-di-tert-butylsalicylidene)-1,2-phenylenediaminoaluminum cobalt tetracarbonyl [(salen)Al(THF)₂]⁺[Co(CO)₄]⁻; Bis(tetrahydrofuran)-meso-tetraphenylporphyrinato aluminum tetracarbonyl cobaltate, [(TPP)Al(THF)₂]⁺[Co(CO)₄]⁻; Bis(tetrahydrofuran)-meso-tetra(4-chlorophenyl)porphyrinato aluminum tetracarbonyl cobaltate, [(4-ClTPP)Al(THF)₂]⁺[Co(CO)₄]⁻; Bis(tetrahydrofuran)-meso-tetra(4-methoxyphenyl)porphyrinato aluminum tetracarbonyl cobaltate, [(4-OMeTPP)Al(THF)₂]⁺[Co(CO)₄]⁻; Bis(tetrahydrofuran)-meso-tetra(2,4,6-trimethylphenyl)porphyrinato aluminum tetracarbonyl cobaltate, [(2,4,6-trimethylTPP)Al(THF)₂]⁺[Co(CO)₄]⁻; Bis(tetrahydrofuran)-octaethylporphyrinato aluminum tetracarbonyl cobaltate, [(OEP)Al(THF)₂]⁺[Co(CO)₄]⁻.

In various examples, one or more, substantially all, or all of the catalyst(s) is/are chiral carbonylation catalysts (e.g., one or more, substantially all, or all of the catalyst(s) comprise(s) a chiral Lewis acid or the like, such as, for example, a Lewis acid comprising one or more chiral ligands or the like). In various examples, at least one, substantially all, or all of the Lewis acid cations (e.g., Lewis acid cations in a catalyst) are chiral Lewis acid cations. Without intending to be bound by any particular theory, it is considered that when a reaction mixture comprises a chiral catalyst or catalysts, carbonylation of the 2,2,3-trisubstituted epoxide(s) can be enantioselective. In various examples, a particular enantiomer or particular enantiomers of the trisubstituted lactone(s) (e.g., 3,3,4-trisubstituted β-lactone(s), such as, for example, 3,3,4-trialkyl-2-oxetanone(s) (e.g., 3,3,4-trimethyl-2-oxetanone and the like) and the like, and any combination thereof) is/are produced.

A reaction mixture (e.g., of a method) can comprise various catalyst concentrations. In various examples, one or more catalyst(s) is/are present (e.g., independently or in the aggregate) in a reaction mixture at a concentration of about 0.0001% to about 1 mol %, including all 0.00001 mol % values and ranges therebetween (e.g., about 0.0001 to about 0.1 mol %, about 0.001 to about 1 mol %, about 0.05 to about 1 mol %, about 0.01 mol %) The mol % is relative to the amount (e.g., moles) of 2,2,3-trisubstituted epoxide(s) in the reaction mixture.

A reaction mixture (e.g., of a method) may further comprise one or more solvent(s). Various solvents can be used. In various examples, a reaction mixture comprises one solvent. In various examples, a reaction mixture comprises a combination of two or more solvents. Non-limiting examples of solvents include ethereal solvents, such as, for example, THF, 2-methyltetrahydrofuran, 2,5-dimethyltetrahydrofuran, 1,3-dioxane, 1,4-dioxane, alkyl ethers (e.g., diethylether, diisopropyl ether, and the like), benzene, toluene, ⁱPr₂O, and the like, acetates (e.g., ethyl acetate, ^{t}butyl acetate, and the like) and the like, and any combination thereof. In various examples, a solvent comprises or is a non-chlorinated, aprotic solvent. In various examples, a reaction mixture comprises THF. In various examples, a reaction mixture comprises THE and 1,4-dioxane. In various examples, one or more solvent(s) in the reaction mixture has/have a concentration of about 0.10 M to about 12 M, including every 0.01 M value and range therebetween (e.g., about 0.5 M), with respect to the 2,2,3-trisubstituted epoxide(s) (e.g., the moles of 2,2,3-trisubstituted. In various examples, a reaction mixture does not comprise a solvent.

A method can be carried out at various reaction pressures. In various examples, a reaction mixture is pressurized to about 0.1 to about 2000 pounds per square inch gauge (psig), including every 0.1 psig value and range therebetween (e.g., about 900 psig). In various examples, a reaction mixture is pressurized to about 0.1 to about 2000 pounds per square inch (psi), including all 0.1 psi values and ranges therebetween (e.g., about 800 psi, about 900 psi, and the like). In various examples, a reaction mixture is pressurized by a gas, such as, for example, CO gas.

A method can be carried out at various reaction temperatures. In various examples, a reaction mixture is heated (e.g., with or without an exogeneous heat source). In various examples, a reaction temperature is about −50° C. to about 200° C., including every 0.1° C. value and range therebetween, such as, for example, about 18° C. to about 25° C., about 24° C., or the like.

A method can be carried out for various reaction times. The reaction time can depend on factors such as, for example, temperature, pressure, presence and/or efficiency of a catalyst and/or activator, presence and/or intensity of an applied energy source, mixing (e.g., stirring or the like), or the like, or a combination thereof. In various examples, reaction times range from about 1 minute (m or min) to about 96 hours (h or hr), including every second value and range therebetween. In various examples, a reaction mixture is held for a time sufficient to achieve a desired trisubstituted lactone composition, structure, yield, or the like, or any combination thereof.

In various examples, a method further comprises isolation of a reaction product (e.g., a trisubstituted lactone) from a reaction mixture. In various examples, one or more trisubstituted lactone(s) prepared by a method as described herein is/are isolated. Suitable isolation methods are known in the art.

In various examples, reactions (e.g., of a method of the present disclosure) have desirable turnover frequencies ((mol starting material consumed)/[(mol catalyst)·(tᵣₓₙ in hours)]). In various examples, reactions have a turnover frequency of about 1 to about 10,000, including all integer values and ranges therebetween (e.g., about 1 to about 1000, about 1 to about 500, about 1 to about 100, about 1 to about 50, about 1 to about 20, or the like).

In various examples, reactions (e.g., of a method of the present disclosure) have desirable turnover numbers. In various examples, reactions have a turnover number of about 1 to about 10,000, including all integer values and ranges therebetween (e.g., about 1 to about 1000, about 1 to about 500, about 1 to about 100, about 1 to about 50, about 1 to about 20, or the like).

In an aspect, the present disclosure provides trisubstituted lactones ((e.g., 3,3,4-trisubstituted β-lactone(s) (such as, for example, 3,3,4-trialkyl-2-oxetanone(s) (e.g., 3,3,4-trimethyl-2-oxetanone and the like) and the like, and any combination thereof)). In various examples, a trisubstituted lactone or trisubstituted lactones (e.g., 3,3,4-trisubstituted β-lactone(s) (such as, for example, 3,3,4-trialkyl-2-oxetanone(s) (e.g., 3,3,4-trimethyl-2-oxetanone and the like) and the like, and any combination thereof)) are produced by a method of the present disclosure. Non-limiting examples of trisubstituted lactones are provided herein.

In various examples, a trisubstituted lactone (e.g., a 3,3,4-trisubstituted β-lactone (such as, for example, 3,3,4-trialkyl-2-oxetanone (e.g., 3,3,4-trimethyl-2-oxetanone or the like) or the like) comprises the following structure:

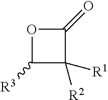

(such as, for example,

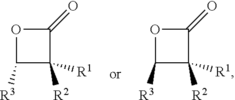

or the like, or any combination thereof) or the like, or a structural analog thereof, or a stereoisomer thereof, or any combination thereof. In various examples, R¹, R², and R³ are, independently at each occurrence, a substituted or unsubstituted alkyl group. In various examples, R¹, R², and R³ comprise or are the same substituted or unsubstituted alkyl groups (e.g., methyl groups, ethyl groups, or the like). In various examples, any one or more of R¹, R², and R³ comprises or is a different alkyl group from the other R group(s). In various examples, any two of R¹, R², and R³ (e.g., R¹ and R², R¹ and R³, or R² and R³) are bonded (e.g., covalently bonded or the like) such that they form a ring (e.g., a substituted or unsubstituted carbocycle, a substituted or unsubstituted heterocycle, or the like).

In various examples, a trisubstituted lactone (e.g., a 3,3,4-trisubstituted β-lactone (such as, for example, 3,3,4-trialkyl-2-oxetanone or the like) or the like) comprises the following structure:

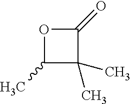

(such as, for example,

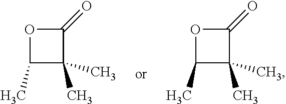

or the like, or any combination thereof) or the like, or a structural analog thereof, or a stereoisomer thereof, or any combination thereof.

In various examples, a trisubstituted lactone (e.g., a 3,3,4-trisubstituted β-lactone (such as, for example, 3,3,4-trialkyl-2-oxetanone or the like) or the like) comprises the following structure:

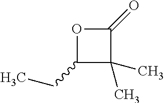

(such as, for example,

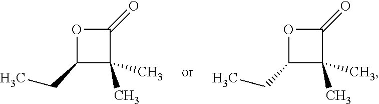

or the like, or any combination thereof) or the like, or a structural analog thereof, or a stereoisomer thereof, or any combination thereof.

In various examples, a composition (which may be a composition suitable for use in a polymerization) comprises one or more lactone(s) (e.g., 3,3,4-trisubstituted β-lactone(s) (such as, for example, 3,3,4-trialkyl-2-oxetanone(s) (e.g., 3,3,4-trimethyl-2-oxetanone, 4-ethyl-3,3,-dimethyloxetan-2-one, and the like) and the like, and any combination thereof)). In various examples, all or substantially all the lactone(s) in a composition is/are 3,3,4-trialkyl-2-oxetanone(s). In various examples, the composition has not been subjected to any purification and/or separation process(s) (such as, for example, enantioselective purification and/or process(es) or the like). Non-limiting examples of purification and/or separation process(s), which may be enantioselective purification and/or process(es), include chromatography, crystallization, and the like, and any combination thereof. In various examples, the composition comprises one or more lactones as synthesized (e.g., the lactone(s) has/have not been subjected to any purification and/or separation process(s) (such as, for example, enantioselective purification and/or separation process(es) or the like).

In various examples, lactone(s) are able to undergo polymerization (e.g., by a ring-opening reaction). Suitable polymerization methods are known in the art. In various examples, a polymer (e.g., a polymer formed from one or more trisubstituted lactone(s) or the like) comprises the following structure:

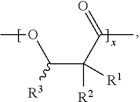

where x is the number of monomer(s) in the polymer chain (such as, for example,

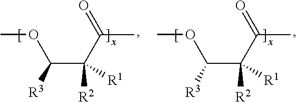

or the like) or the like, or a structural analog thereof, or a stereoisomer thereof, or any combination thereof.

In various examples, a polymer (e.g., a polymer formed from one or more trisubstituted lactone(s) or the like) comprises the following structure:

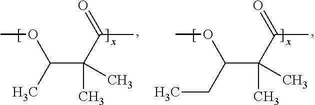

or the like, or a structural analog thereof, or a stereoisomer thereof, or any combination thereof.

The following Statements describe various examples of trisubstituted lactones and methods of making same that are not intended to be limiting in any manner.

Statement 1. A method of producing one or more lactone(s) (e.g., 3,3,4-trisubstituted β-lactone(s) (such as, for example, 3,3,4-trialkyl-2-oxetanone(s) (e.g., 3,3,4-trimethyl-2-oxetanone and the like) and the like, and any combination thereof)), comprising: providing a reaction mixture comprising one or more 2,2,3-trisubstituted epoxide(s) or the like (such as, for example, one or more racemic mixture(s) of 2,2,3-trisubstituted epoxide(s) or the like, one or more enantiomer(s) of 2,2,3-trisubstituted epoxide(s) or the like, or the like, or any combination thereof), one or more carbonylation catalyst(s) or the like in a vessel (e.g., a sealed vessel), optionally, with a solvent or two or more solvents (e.g., a polar, aprotic solvent or two or more polar, aprotic solvents, or the like, or any combination thereof); contacting (e.g., pressurizing, sparging, or the like, or any combination thereof) the reaction mixture with carbon monoxide; and holding the reaction mixture (e.g., stirring the reaction mixture or the like) for a selected time (1 minute (m or min) to 96 hour (h or hr)) and/or temperature (−50 to 200° C. (e.g., 18 to 25° C., such as, for example, 24° C.)) where the trisubstituted lactone(s) (e.g., 3,3,4-trisubstituted β-lactone(s) (such as, for example, 3,3,4-trialkyl-2-oxetanone(s) (e.g., 3,3,4-trimethyl-2-oxetanone and the like) and the like, and any combination thereof)) is/are produced.
Statement 2. A method according to Statement 1, where the reaction mixture is vented.
Statement 3. A method according to Statement 1 or Statement 2, where the trisubstituted lactone(s) is/are isolated.
Statement 4. A method according to any one of Statements 1-4, where a catalyst or one or more or all of the catalyst(s) is/are, independently, preformed or formed in situ (e.g., from a reaction with one or more catalytic precursor(s) and one or more metal source(s), such as, for example, a cobalt metal source (e.g., NaCo(CO)₄ or the like, or any combination thereof)).
Statement 5. A method according to any one of the preceding Statements, where the catalyst(s) is/are present (independently or in the aggregate) in the reaction mixture at a concentration of about 0.0001 to about 1 mol % (e.g., about 0.001 to about 0.1 mol %, about 0.001 to about 1 mol %, about 0.05 to about 1 mol %, about 0.01 mol %).
Statement 6. A method according to any one of the preceding Statements, where the solvent(s) is/are chosen from hydrocarbons, THf, 1,3-dioxane, 1,4-dioxane, diethyl ether, toluene, ⁱPr₂O, ethyl acetate, and the like, and structural analogs thereof, and any combination thereof.
Statement 7. A method according to any one of the preceding Statements, where the solvent(s) is/are present in the reaction mixture at about 0.10 to about 12 M, including every 0.01 M value and range therebetween (e.g., about 0.5 M), with respect to the 2,2,3-trisubstituted epoxide or the like.
Statement 8. A method according to any one of the preceding Statements, where the reaction mixture is pressurized to about 0.1 to about 2000 psig (e.g., about 900 psig) with carbon monoxide (CO).
Statement 9. A method according to any one of the preceding Statements, where the carbonylation catalyst(s) is/are Lewis Acid catalyst(s) (such as, for example, Lewis Acid carbonylation catalysts or the like) or the like.
Statement 10. A method according to Statement 9, wherein the carbonylation catalyst(s) independently comprise(s) a cationic Lewis acid and an anionic metal carbonyl.
Statement 11. A method according to Statement 9, wherein the Lewis acid carbonylation catalyst(s) independently comprise a salen ligand, a porphyrin ligand, a structural analog thereof, or the like.
Statement 12. A method according to Statement 10 or 11, wherein the Lewis acid carbonylation catalyst(s) independently comprise an Al³⁺ cation, a Cr³⁺ cation, or the like.
Statement 13. A method according to Statements 10 or 12, wherein anionic metal carbonyl is [Co(CO)₄]⁻, a structural analog thereof, or the like.
Statement 14. A method according to any one of the preceding Statements, where the method is carried out in a single vessel (e.g., without separating or isolating various possible intermediates, which may be referred to as a “one-pot reaction”).
Statement 15. A method according to any one of the preceding Statements, where the 2,2,3-trisubstituted epoxide(s) independently comprise(s) the following structure:

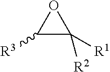

(such as, for example

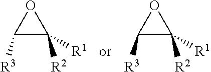

or a mixture thereof) or the like, or a structural analog thereof, or a stereoisomer thereof, or any combination thereof, where R¹, R², and R³ are, independently at each occurrence, a substituted or unsubstituted alkyl group that is the same or different than the other R groups, or R¹ and R² or R¹ and R³, or R² and R³ are connected such that they form a substituted or unsubstituted carbocycle or substituted or unsubstituted heterocycle.
Statement 16. A method according to Statement 15, where the substituted or unsubstituted alkyl groups, independently at each occurrence, comprise linear or branching alkyl groups or cyclic alkyl groups.
Statement 17. A method according to Statement 16, where the linear or branching alkyl groups, independently at each occurrence, comprise a longest linear chain of 1 to 12 carbons (e.g., 1, 2, 3, 4, 5, 6, 7, 8, 9, 10, 11, or 12 carbons).
Statement 18. A method according to any one of Statements 15-17, where R¹, R², and R³ are each independently chosen from substituted or unsubstituted methyl groups and substituted or unsubstituted ethyl groups.
Statement 19. A method according to any one of Statements 15-18, where R¹, R², and R³ comprise substituted or unsubstituted methyl groups.
Statement 20. A method according to any one of Statements 15-18, where R¹ and R² each comprise a substituted or unsubstituted methyl group, and R³ comprises a substituted or unsubstituted methyl group or a substituted or unsubstituted ethyl group.
Statement 21. A method according to any one of the preceding Statements, where the trisubstituted lactone(s) comprise(s) one or more 3,3,4-trisubstituted β-lactone(s).
Statement 22. A method according to Statement 21, where the 3,3,4-trisubstituted β-lactone(s) (such as, for example, 3,3,4-trialkyl-2-oxetanone(s) and the like, and any combination thereof) independently comprise(s) the following structure:

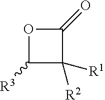

(such as, for example,

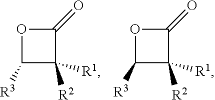

or a mixture thereof), wherein R¹, R², and R³ are, independently at each occurrence, a substituted or unsubstituted alkyl group, or R¹ and R², R¹ and R³, or R² and R³ are connected such that they form a substituted or unsubstituted carbocycle or substituted or unsubstituted heterocycle, or the like, or a structural analog thereof, or a stereoisomer thereof, or any combination thereof.
Statement 23. A method according to any one of the preceding Statements, where the lactone(s) is/are the result of a carbonylation at the more substituted position on the 2,2,3-trisubstituted epoxide(s).
Statement 24. A method according to Statement 22, where the substituted or unsubstituted alkyl groups, independently at each occurrence, comprise linear or branching alkyl groups or cyclic alkyl groups.
Statement 25. A method according to Statement 24, where the linear or branching alkyl groups, independently at each occurrence, comprise a longest linear chain of 1 to 12 carbons.
Statement 26. A method according to any one of Statements 22-25, where R¹, R², and R³ are each independently chosen from substituted or unsubstituted methyl groups and substituted or unsubstituted ethyl groups.
Statement 27. A method according to any one of Statements 22-26, where R¹, R², and R³ comprise substituted or unsubstituted methyl groups.
Statement 28. A method according to any one of Statements 22-26, where R¹ and R² each comprise a substituted or unsubstituted methyl group, and R³ comprises a substituted or unsubstituted methyl group or a substituted or unsubstituted ethyl group.
Statement 29. A method according to any one of Statements 21-28, where the 3,3,4-trisubstituted β-lactone(s) independently comprise(s) the following structure:

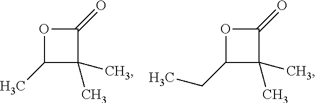

or any stereoisomer thereof, or the like.
Statement 30. A composition, comprising one or more trisubstituted lactone(s) formed by a method according to any one of the preceding Statements.
Statement 31. A polymer formed from one or more trisubstituted lactone(s) formed by a method of any one of Statements 1-29.

The steps of the method described in the various examples disclosed herein are sufficient to carry out the methods of the present disclosure. Thus, in various examples, a method consists essentially of a combination of the steps of the methods disclosed herein. In various other examples, a method consists of such steps.

The following example is presented to illustrate the present disclosure. It is not intended to be limiting in any manner.

### Example

The example describes an example of a method of the present disclosure.

Bis(tetrahydrofuran)-meso-tetraphenylporphyrinato aluminum tetracarbonyl cobaltate, [(TPP)Al(THF)₂]⁺[Co(CO)₄]⁻ was prepared by methods known in the art.

In a glovebox, a 4 mL glass vial equipped with a Teflon-coated magnetic stir bar and septum cap was charged with 7.6 mg of [(TPP)Al(THF)₂]⁺[Co(CO)₄]⁻, 0.50 ml of 1,4-dioxane, and 44.2 mg of racemic 2,2,3-trimethyloxirane. The contents were placed in a high-pressure reactor. The reactor was subsequently sealed, taken out of the glovebox, placed in a well-ventilated hood, and pressurized with carbon monoxide (900 psi). The closed reactor stirred at 60° C. for 3 hours. The reactor was carefully vented, and the contents were analyzed by ¹H NMR spectroscopy. The epoxide was completely consumed, and the only observable product was racemic 3,3,4-trimethyl-2-oxetanone.

Although the present disclosure has been described with respect to one or more particular example(s), it will be understood that other examples of the present disclosure may be made without departing from the scope of the present disclosure.

1. A method of producing one or more trisubstituted lactone(s), comprising:
providing a reaction mixture comprising one or more 2,2,3-trisubstituted epoxide(s), one or more catalyst(s), and, optionally, one or more solvent(s);
contacting the reaction mixture with carbon monoxide (CO); and,
holding the reaction mixture for a selected time and/or at a selected pressure and/or temperature, wherein the trisubstituted lactone(s) is/are produced.

2. The method of claim 1, wherein the reaction mixture is vented.

3. The method of claim 1, wherein the trisubstituted lactone(s) is/are isolated.

4. The method of claim 1, wherein the one or more catalyst(s) is/are independently formed in situ.

5. The method of claim 1, wherein the catalyst(s) is/are present in the reaction mixture at a concentration of about 0.0001 mol % to about 1 mol % based on total 2,2,3-trisubstituted epoxide(s) in the reaction mixture.

6. The method of claim 1, wherein the solvent(s) is/are independently chosen from hydrocarbons, THF, 1,3-dioxane, 1,4-dioxane, diethylether, diisopropyl ether, benzene, toluene, ⁱPr₂O, ethyl acetate, and structural analogs thereof, and any combination thereof.

7. The method of claim 1, wherein the solvent(s) is/are present in the reaction mixture at a concentration of about 0.10 M to about 12 M.

8. The method of claim 1, wherein the reaction mixture is pressurized to about 0.1 to about 2000 psi with the carbon monoxide (CO).

9. The method of claim 1, wherein one or more of the catalyst(s) independently comprise(s) one or more Lewis Acid catalyst(s).

10. The method of claim 9, wherein one or more of the catalyst(s) independently comprise(s) one or more cationic Lewis acid(s) and one or more anionic metal carbonyl(s).

11. The method of claim 9, wherein one or more of the Lewis acid catalyst(s) independently comprise(s) a salen ligand, a porphyrin ligand, a metallocene ligand, a structural analog thereof, or any combination thereof.

12. The method of claim 9, wherein one or more of the Lewis acid catalyst(s) independently comprise(s) an Al³⁺ cation or a Cr³⁺ cation.

13. The method of claim 10, wherein anionic metal carbonyl is [Co(CO)₄]⁻.

14. The method of claim 1, wherein the method is carried out in a single vessel.

15. The method of claim 1, wherein the 2,2,3-trisubstituted epoxide(s) independently comprise(s) the following structure: 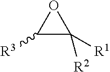
or a structural analog thereof, or a stereoisomer thereof, or any combination thereof,
wherein R¹, R², and R³ are, independently at each occurrence, a substituted or unsubstituted alkyl group, or R¹ and R², R¹ and R³, or R² and R³ are connected such that they form a substituted or unsubstituted carbocycle or substituted or unsubstituted heterocycle.

16. The method of claim 15, wherein the 2,2,3-trisubstituted epoxide(s) comprise(s) 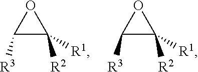
or a mixture thereof.

17. The method of claim 15, wherein the substituted or unsubstituted alkyl groups, independently at each occurrence, comprise linear or branching alkyl groups or cyclic alkyl groups.

18. The method of claim 17, wherein the linear or branching alkyl groups, independently at each occurrence, comprise a longest linear chain of 1 to 12 carbons.

19. The method of claim 15, wherein R¹, R², and R³ are each independently chosen from substituted or unsubstituted methyl groups and substituted or unsubstituted ethyl groups.

20. The method of claim 15, wherein R¹, R², and R³ comprise substituted or unsubstituted methyl groups.

21. The method of claim 15, wherein R¹ and R² each comprise a substituted or unsubstituted methyl group, and R³ comprises a substituted or unsubstituted methyl group or a substituted or unsubstituted ethyl group.

22. The method of claim 1, where the trisubstituted lactone(s) comprise(s) one or more 3,3,4-trisubstituted β-lactone(s).

23. The method of claim 22, wherein the 3,3,4-trisubstituted β-lactone(s) independently comprise(s) the following structure: 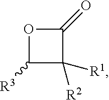
wherein R¹, R², and R³ are, independently at each occurrence, a substituted or unsubstituted alkyl group, or R¹ and R², R¹ and R³, or R² and R³ are connected such that they form a substituted or unsubstituted carbocycle or substituted or unsubstituted heterocycle.

24. The method of claim 23, wherein the 3,3,4-trisubstituted β-lactone(s) independently comprise(s) 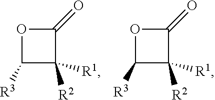
or a mixture thereof.

25. The method of claim 23, wherein the substituted or unsubstituted alkyl groups, independently at each occurrence, comprise linear or branching alkyl groups or cyclic alkyl groups.

26. The method of claim 23, wherein the linear or branching alkyl groups, independently at each occurrence, comprise a longest linear chain of 1 to 12 carbons.

27. The method of claim 23, wherein R¹, R², and R³ are each independently chosen from substituted or unsubstituted methyl groups and substituted or unsubstituted ethyl groups.

28. The method of claim 23, wherein R¹, R², and R³ comprise substituted or unsubstituted methyl groups.

29. The method of claim 23, wherein R¹ and R² each comprise a substituted or unsubstituted methyl group, and R³ comprises a substituted or unsubstituted methyl group or a substituted or unsubstituted ethyl group.

30. The method of claim 22, wherein the 3,3,4-trisubstituted β-lactone(s) independently comprise(s) the following structure: 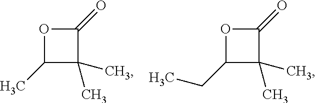
or any stereoisomer thereof.

31. A composition, comprising one or more trisubstituted lactone(s) formed by the method of claim 1.

32. A polymer formed from one or more trisubstituted lactone(s) formed by the method of claim 1.
